(19) United States
(12) Patent Application Publication
(10) Pub. No.: US 2021/0120369 A1
(43) Pub. Date: Apr. 22, 2021

(54) INDOOR POSITIONING SYSTEM AND METHOD

(71) Applicant: J2SQUARE, LLC, Burlington, NJ (US)

(72) Inventor: Fernando J. PINHO, Burlington, NJ (US)

(21) Appl. No.: 17/081,039

(22) Filed: Oct. 27, 2020

Related U.S. Application Data
(63) Continuation-in-part of application No. 16/205,551, filed on Nov. 30, 2018.
(60) Provisional application No. 62/594,156, filed on Dec. 4, 2017.

(51) Int. Cl. H04W 4/029; H04W 4/02; H04W 4/33; A61B 5/0205; A61B 5/00; G06F 1/16; A61B 5/24

(52) U.S. Cl. CPC H04W 4/029; H04W 4/023; H04W 4/026; H04W 4/027; A61B 5/24; A61B 5/02055; A61B 5/6802; G06F 1/163; A61B 5/0002; H04W 4/33

(57) ABSTRACT

An improved positioning and navigational system for indoor use providing reliable positioning signals in a cost effective manner for use in construction projects, in the building trades, and inspection businesses, activities that relate to using surveys, floorplans, and blueprints, and security systems for a variety of buildings, such as schools, government buildings, apartment buildings, and office building, which system includes sensors for monitoring and recording the proximities between persons working at, inspecting, or visiting such buildings and their body temperatures and other vital signs.

### CROSS REFERENCE TO RELATED APPLICATION

This is a continuation in part application based on pending U.S. Utility application Ser. No. 16/205,551 that claimed priority from U.S. Provisional Application No. 62/594,156 filed on Dec. 4, 2017, and follows on U.S. Provisional Applications Nos. 62/352,598 and 62/423,349 filed on Jun. 21, 2016 and Nov. 17, 2016 respectively and pending U.S. Utility application Ser. No. 15/628,700 filed on Jun. 21, 2017, all of which being incorporated by reference herein.

### FIELD OF THE INVENTION

The present invention relates positioning systems for indoor use including: (i) systems for use in construction projects, in the building trades, and inspection businesses; (ii) activities that relate to using surveys, floorplans, and blueprints, and (iii) security systems for a variety of buildings, such as schools, government buildings, apartment buildings, and office buildings, as well as to a panoply of other uses that require tracking, security, and related tasks in interior spaces.

### BACKGROUND OF THE INVENTION

While systems based on satellite-based radio navigation system known as the Global Positioning System, or GPS, are used in a wide variety of applications, GPS-based indoor positioning applications suffer from the limited reception of the relatively weak signals that emanate from distant satellites within solid structures, to say nothing of tunnels, or even under dense cloud cover. The problem to be solved by the instant invention is to provide reliable positioning signals, other than by use of GPS, within solid structures in a cost effective manner. While indoor systems that require a panoply of expensive, ubiquitous, and redundant hardware platforms are in use in the public domain, the present invention is based on a self-contained, cost effective system that eschews such redundant, and hardware dependent, systems, such as beacons. That invention is based on the use of cost effective, limited hardware, that is, the use of a portable, electronic device, an indoor navigation device, or “IND,” in conjunction with a smartphone running an application program keyed to the structure involved, such as the program outlined in the co-pending utility application referenced hereinabove. In addition, the present application relates to an improvement to said navigation device that allows for the monitoring and recording of the proximities between persons working at an indoor construction project, or inspectors of such a project, or persons working in or visiting buildings, as well as the body temperatures and other vital signs of such workers or other persons.

### SUMMARY OF THE INVENTION

The indoor navigation device, or IND, of the present disclosure is a portable electronic device, smaller in size than a handheld, specifically developed for indoor navigation and positioning in situations in which there is limited or no access to signals emanating from the Global Positioning System. The device is based on the use of an electromechanical unit that comprises an accelerometer, a gyroscope, and a compass described with more detail hereinbelow. The device, having its own self-contained power source, functions independently as those sensors are embedded within the device itself, and needs no external hardware accessories, such as beacons, for the device to function as a locator and positioning tool without resort to GPS signals. An embedded microprocessor in the IND processes raw sensor data from those sensors when the device is moved by the user and coordinates are continually updated for each displacement of the device. Latest sensor values are transmitted continuously through a Bluetooth® interface using Bluetooth® Low Energy technology (“BLE”) to a receiving and processing device, such as smartphone. The receiving device is Bluetooth paired with IND before starting to receive data from the sensors. Device size is minimized by the use of highly compact size of microelectromechanical (MEMS) technology that provides sensor values in direct digital formats.

In this way, the entire system for indoor positioning consists only of two relatively small devices working in coordination with each other.

For those working in construction projects, or inspecting buildings in the process of construction or after construction has been long completed, or those entering into buildings for any purpose, such as, without limitation, first responders, teachers, students, or administrators, tracking the proximity of one person to another has become an important problem to be solved since the advent of the current pandemic. This invention solves that problem by the use of the portable, wearable electronic devices described herein.

In a preferred embodiment, this invention solves the problem of tracking the proximity between workers at a construction project site, which proximity has become important in the face of a global pandemic for a highly contagious virus, as well as the body temperature of said workers and other vital signs.

### BRIEF DESCRIPTION OF THE DRAWINGS

FIG. 1 is a representation of the relationship between the main components of the system of the present invention.

FIG. 2 is a block diagram showing the main components of the indoor navigation device of the present invention.

FIG. 3 is a perspective drawing of one of the components of the indoor navigation device of the present invention indicating the axes measured by such component.

FIG. 4 is a flow chart for operation of the system of the present invention.

FIG. 5 is a perspective drawing of the indoor navigation device of the present invention indicating the approximate measurements of said device.

FIG. 6 is shows the system of the present invention in use in a construction setting.

FIG. 7 comprises a series of drawings illustrating three embodiments of the indoor navigation device with improvements, to wit, FIG. 7A is a front view of such a device and a wrist mounting strap; FIG. 7B is a front view of such a device with a chest mounting strap; and FIG. 7C is the front view of such a device for mounting on a construction worker helmet.

FIG. 8 is a block diagram showing certain components of the device of FIG. 7A.

FIG. 9 is a block diagram showing certain components of the device of FIG. 7A.

FIG. 10 is a floor plan of a building showing the device of FIG. 7A located in the lobby of said building.

### DETAILED DESCRIPTION OF THE INVENTION

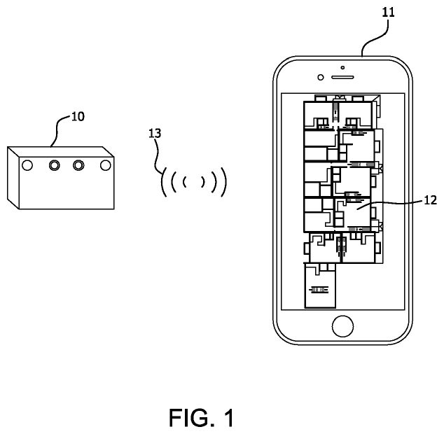

FIG. 1 shows the indoor navigation device, or IND, 10 of the present disclosure, a portable electronic device, smaller in size than a handheld, specifically developed for indoor navigation and positioning in situations in which there is limited or no access to signals emanating from the Global Positioning System. The device is based on the use of an electromechanical unit that comprises an accelerometer, a gyroscope, and compass, has a self-contained power source, and uses an embedded microprocessor to process raw sensor data from those sensors when the device is moved by the user. IND device 10 coordinates with the wireless capable handheld device 11, such as a smartphone running an application program keyed to the structure involved as shown by the electronic floor plan 12 displayed on the touchscreen of the device in FIG. 1. Sensor values are transmitted continuously via radio-frequency (RF) technology 13, such a wireless Bluetooth® interface 13 using Bluetooth® Low Energy technology (“BLE”), to the receiving and processing device, such as smartphone 11. The receiving device 11 is Bluetooth paired with IND 10, receiving an initializing signal from device 11, before starting to process data from its components. Device 10 size is minimized by the use of highly compact size of microelectromechanical (MEMS) technology that provides sensor values in direct digital formats. In this way, the entire hardware system for indoor positioning consists only of two handheld devices, IND 10 and smartphone 11, working in wireless coordination.

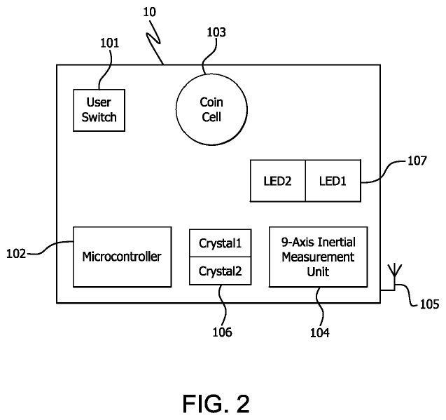

FIG. 2 is a block diagram showing the components of IND 10. User switch 101 is a push button type of switch for waking up microcontroller 102 from its power saving “sleep mode.” When this switch is pressed by the user, microcontroller 102 wakes up and, as explained below, broadcasts a wireless identification signal for discovery by receiving device 11 and waits for an initializing connection from such device 11 running the applicable electronic floorplan 12 application based on the position of the user as initialized on the touch screen of said electronic floorplan. If no connection request is received from such device 11 within ten seconds, the microcontroller 102 will go to sleep again to save power. In the preferred embodiment of device 10, the microcontroller 102 selected is chip CC2650, a wireless Micro Controller Unit (MCU) of CC26XX family of microcontrollers from Texas Instruments, which supports multiple wireless protocols in 2.4 GHz frequency range, such as BLE, ZigBee, 6LowPAN, and ZigBee RF4CE. This chip is cost-effective and power efficient for use in battery operated devices and contains 32-bit ARM Cortex-M3 operating at 48 MHz as the main processor and has rich set of peripherals. Constructed in this way, device 10 utilizes a dedicated ARM Cortex-MO for running BLE under IEEE 802.15.4 MAC protocol. This architecture improves overall system performance and power consumption and frees up flash memory for the application. The chip 102 of the preferred embodiment supports 128 kB of programmable flash, 8-kB SRAM for cache and 20 kB of Ultra-low leakage SRAM and supports peripherals like GPIOs, Timers, UART, 12C, SPI, Real Time Clock (RTC), AES-128 security module, and True Random Number Generator (TRNG).

IND 10 is powered by a self-contained power source 103, such as a 3.0 volts CR2032 coin cell battery in the preferred embodiment situated in a coin cell battery holder by which a user can insert and remove the battery easily. IND 10 is outfitted with two light signaling elements 107: LED1 is a connection indicator, that is, in the preferred embodiment, a red color SMD LED that that keeps blinking every second while device 10 waits for an initializing connection from the floorplan 12 navigator application running on smartphone 11. When a connection is established with smartphone 11, LED1 blinks 5 times with 300 milliseconds gap and goes off in the preferred embodiment. The second light signaling element in element 107 is LED2, a green color SMD LED, that blinks every time a position value is shared with the floor navigator application in receiver 11, indicating to the user the successful receipt by receiver 11 of positioning values via wireless signals 13 for processing by the floorplan 12 navigator application program.

As shown in FIG. 2, device 10 is further comprised of component element 106 which contains two crystals: crystal 1 in the preferred embodiment is an SMD type 24 MHz crystal oscillator that is used to drive the main operating clock of microcontroller chip 102 and crystal 2 is SMD type 32.768 kHz crystal oscillator that is used to drive the real time clock of the microcontroller chip 102.

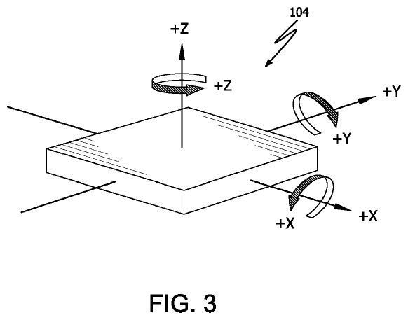

IND device 10 includes element 104, a nine-axis inertial measurement unit 104, which, in the preferred embodiment, is selected to be MPU9250, a multi-chip module that houses a 3-axis accelerometer, 3-axis gyroscope, and 3-axis magnetometer. FIG. 3 provides a view of the nine-axes that are sampled by measurement unit 104 as the X, Y, Z axes of sensitivity and polarity or rotation shown. As well as having the 3-axis gyroscope, 3-axis accelerometer, and 3-axis magnetometer, preferred chip MPU9250 provides on chip digital motion processor (DMP) and has a dedicated 12C sensor bus and MPU9250 provides complete 9-axis motion fusion output. This motion tracking device 104, with its 9-axis integration on-chip motion fusion, run-time calibration firmware eliminates costly and complex selection, qualification, and system level integration of discrete components. Preferred MPU9250 features three 16-bit analog to digital converters (ADCs) for digitizing gyroscope analog outputs, three 16-bit ADCs for digitizing accelerometer analog outputs and three 16-bit ADCs for digitizing magnetometer analog outputs. In the preferred embodiment, unit 104 (MPU9250) supports use programmable gyroscope full-scale range of ±250, ±500, ±1000 and ±2000°/sec (dps), a use programmable accelerometer full-scale range of ±2 g, ±4 g, ±8 g, and ±16 g and a magnetometer full scale range of ±4800 μT. The preferred unit 104 operates in the range of 2.4V to 3.6V. Communication with MPU9250 registers is performed using either 12C at 400 kHz or SPI at 1 MHz. The device 10 supports SPI communication at 20 MHz for the applications that requires faster communications. Unit 104, MPU9250, supports in the preferred embodiment nine different user accessible power modes of which Accel+Gyro Mode is used in this system.

Microcontroller chip 102 is provided linear acceleration and angular rotation data from the IMU 104 using an 12C communication on a periodic basis and calculates the new position based the current acceleration and rotation data and position information. The newly calculated position data as determined by microcontroller 102 is sent to the mobile application running on smartphone 11 and location then being displayed on the electronic floor map 12 of the current floor plan under navigation application miming on smartphone 11.

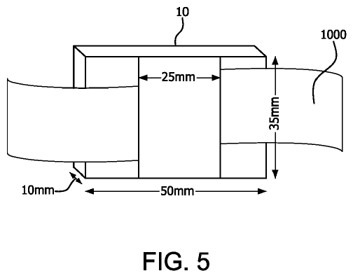

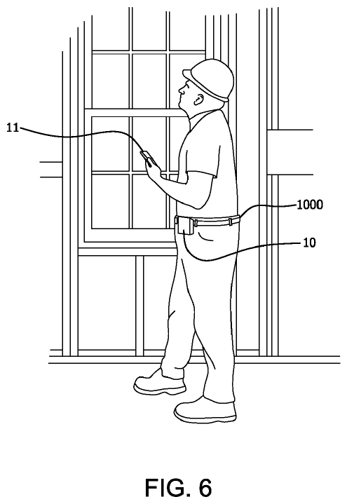

As can be appreciated by those skilled in the art, device 10 is also comprised of additional electronic components, such as a printed circuit board, resistors, capacitors, and diodes of the electronic circuitry that help in filtering noise in the power supply 103, among other things. FIG. 5 is a rear view illustrating the portability and size of IND device 10. A segment of a worker's belt 1000 is shown in FIG. 6 as having been threaded under a belt loop 1001 having a width of 25 mm A preferred embodiment of device 10 is shown in FIG. 5 as having approximate dimensions of 50 mm in length, 35 mm in height, and 10 mm in depth, but as one can appreciate these dimensions can vary in accordance with the designer's needs and desires. FIG. 6 illustrates the invention of the present disclosure in use as device 10 as attached to belt 1000, in the preferred embodiment, of the construction worker on the job who is reviewing location and floorplan 12 information on Bluetooth-connected smartphone 11 held in his hand. By using the belt fastening concept of FIG. 5, the relative position of device 10 is maintained, aiding in accuracy of positioning. As one can appreciate, IND 10 can be fastened to the user in various manners, including, but not limited to, being attached to the shoe or lower leg of the user by appropriate fastening means.

In the disclosed system of the preferred embodiment, chip 102, the CC2650 in device 10, is configured to handle communication with unit 104, MPU9250 unit, over 12C and with mobile application over Bluetooth Smart. The MCU 102, CC2650, is also used for handling user interface actions like switch presses and provide LED indication to the users about the status of the ongoing operations. Device 10 configures unit 104 MPU9250 by writing to the control registers of chip 102 MPU9250 using 12C link and reads the data from MPU9250 using the same 12C link. Microprocessor chip 102 includes Bluetooth Smart stack in the preferred embodiment.

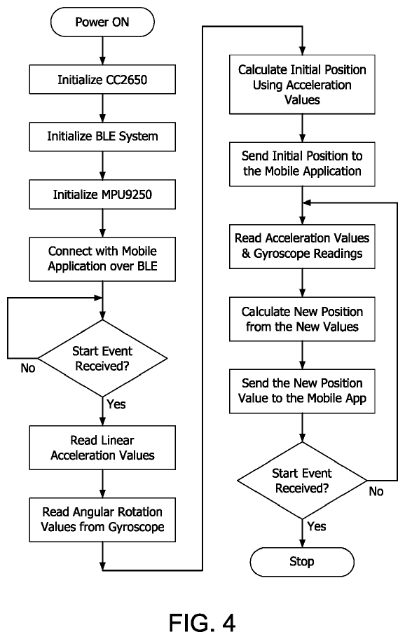

The flowchart of FIG. 4 illustrates the method used in coordinating the hardware of the system of the present disclosure. After being powered on, device 10 initializes all three system components: microcontroller 102, Bluetooth connectivity 13, and the inertial measurement unit 104. Device 10 waits for the position initializing ‘start’ signal from the mobile application and upon receiving the start signal, reads the accelerometer reading and gyroscope reading. The accelerometer and gyroscope readings are in direct digital values which need to be converted to ‘g’ values by multiplying the values received with 9.8 and the result will be an acceleration variable measured in meters/second squared. Based on the accelerometer readings calculate the position and send to the mobile application which, will be considered as the initial position or starting/reference point on the floor map in the mobile application. Device 10 reads the accelerometer and gyroscope readings from unit 104 in IND 10 via RF signals 13 periodically and calculates the new position based on previous position value and recent accelerometer and gyroscope readings. The new position value is sent to the mobile application for displaying it on the floor map 12. New position values are calculated and sent to the mobile application until the user presses ‘stop’ on the mobile application. The new position value is calculated based on acceleration vectors Ax, Ay, Az rotation vectors Gx, Gy, Gz. From these the resultant acceleration vector Racc and rotation vector Rgyro are calculated. New position vector Rnew is calculated using Racc and Rgyro with weights w1, w2 for accelerometer and gyroscope readings. The gyroscope weightage is taken as w2/w1 and is a value between 5 & 20 can be considered based on experimental studies. A value of 12 is considered for Gw in this system. Based on these the new position values PxNew, PyNew, PzNew are calculated and send to the mobile application. The new position values are sent to the application at the rate of one signal every ½ second. The position value updates are sent to the application until the user presses ‘stop’ on the application.

As can be appreciated, the system disclosed can be applied to many uses other than construction projects. The application software of smartphone 11 can be modified for use by authorities, businesses, individuals, and local/county/state governments. Some examples follow:
a. Identifying and making accessible plans, blueprints, layout, and configurations of buildings, properties, businesses, organizations, structures, and homes.
b. Identifying and giving access to individual handheld devices to those persons having access or permission to be in certain buildings, properties, businesses, organizations, structures, and homes. The disclosed system and method will allow for tracking the individual within said buildings and homes as disclosed in the co-pending application.
c. Using device 10 can be used as an personal identifier, key, or locator.
d. Identifying and photographing individuals having access, and storing such individual information within a database specific to a building, property, business, organization, structure, or home, including contact information such as cell phone numbers and email addresses.
e. Using a device 10 in conjunction with facial recognition software in order to:
   1. Identify authorized personnel upon entering the building or business;
   2. Track authorized personnel within the building or business;
   3. Notify authorized person entering the building or business without device 10 or with a malfunctioning device 10 that his or her device 10 is missing or improperly functioning; and
   4. Identify unauthorized personnel immediately.

The following security applications will benefit from the use of the system of the present invention:
1. Implementation of or incorporating an option of a lock down system where no one can enter a room or building, but exiting is always accessible. Access or entry can be given by a person within the room or building, or remotely.
2. Design and manufacturing of a surveillance camera constructed under a smoke/carbon monoxide detectors or constructed to accept the smoke detector under the camera.

As can be appreciated, the disclosed system can be readily applied to planning and zoning uses by local/county/state governments, as the system can be adapted to perform the following tasks:
1. Collect, store, process, maintain, organize, update, forward, and deliver blueprints and plans to be submitted to the zoning authority or local/county/state governments for new and/or previously existing developments, sub-divisions, constructions, building lot, homes, buildings, and improvements of any properties within their boundaries or jurisdiction.
2. Process, collect, store, maintain, organize, update, forward, and deliver documents, plans, applications, reviews, and reports, submitted to or from the zoning authority or local/county/state governments that is relevant to a new or previously existing development, subdivision, construction, building lot, home, building, and improvements of any properties within their boundaries or jurisdiction.
3. The IND can also be modified to serve as a proximity sensor and recorder, that also can sense and record vital signs, such as body temperature and blood oxygen, of worker at a construction project, or inspectors or other persons at any building. To monitor and record proximity of one person to another, the devices can be worn on the wrists, across the chests, or attached to a helmet, or can be worn as a badge, like an ID card or ID badge, or any other device that can located on the person. To monitor vital signs, such as body temperature, oxygen level, or heartbeat, the device would perforce be located on the wrist next to the skin. Most helmets and hard hats come with sweatbands or bands to adjust them o the users head size. Such a proximity monitoring device can be applied to or incorporated in a helmet; however, the device or the sensors that monitor the vital signs (such as, without limitation, body temperature, oxygen level, heartbeat, and perspiration) be attached or incorporated into the sweatband, resulting in very accurate readings, and making the helmet mounted option suitable for providing the full complement of readings that are available in other options. Additionally, the device possibly monitor other body changes, such as coughing.

This system gives employers or managers access to a program or application to monitor employees' devices to make sure that all employees or workers are operating in a compliant environment in accordance with state or local regulations in times of the spread of communicable diseases, such as the flu, a virus, or a ubiquitous pandemic. Other features can also be added to the device such as GPS capabilities, indoor positioning capabilities, and acting as a key or security badge for access to a building.

Additionally, the device can be modified to track and record information relating to areas of indoor spaces that have been physically touched by workers, employees, inspectors, or other persons. Using the indoor tracking capabilities described above for the IND device, or, alternatively, for use with beacons or other sensors strategically placed about an area being monitored helps not only managers, but also, employees returning to work in the face of communicable disease, or visitors to a building in the following ways:
(a) An employer can identify individuals at risk of infection.
(b) The device can make it easier to identify which individuals may have crossed paths.
(c) The employer can possibly identify travel paths later discovered infected employees and take more of a surgical approach to the disinfecting and cleaning of the area.
(d) Employers can track more precisely the areas that need more detailed or concentrated attention when cleaning the premises.
(e) Employers and employees can be notified if someone has been in their private workspace, such as an office or a cubical.
(f) When moving about, employees can be advised as to the activity levels in each area of a building. This can be done by displaying an assortment of different colors representing differing levels of activity or traffic in each area so that employees can avoid areas of greater concentrated activity.
(g) Settings can allow for what duration activity is being monitored, and when the last activity was in the area.

With the added use of GPS in one embodiment, this device can perform as well outdoors as indoors. The preferred embodiment of the device encompasses settings that will allow for options, and with different levels of vibration or sounds. For example, the device can be set to buzz or vibrate to notify the user of another person getting close to him and her, for example, within 10 feet or within 15 feet. If the person gets within a 10 foot radius a progressively louder beeping will be heard the closer the individuals are to each other, followed by a warning that can be set to sound or vibrate at a radius of, for example, 7 feet, or by a voiced alarm of a “Too Close” message.

This device can be employed in a similar manner to alert pedestrians walking about in public of dangerous proximity to others. Bluetooth capabilities within the device can identify other devices typically carried around by individuals with Bluetooth capabilities; the device can identify another person with a cell phone or another devices coining in close proximity. In this way, the preferred embodiment of this mobile device is useable outside of the work environment.

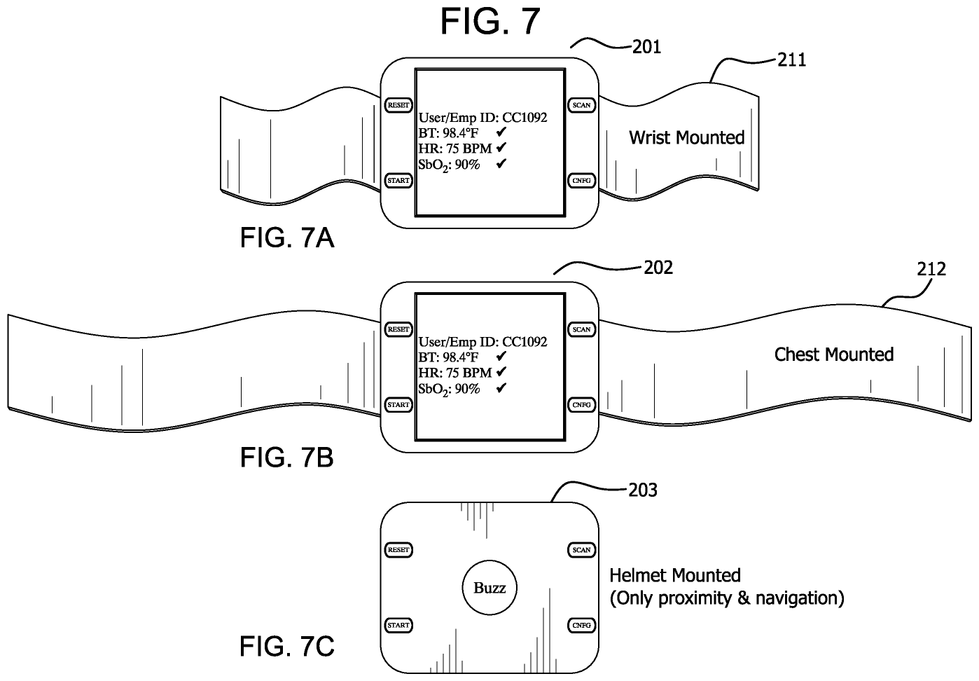

Referring to the drawings, FIG. 7 comprises a series of drawings illustrating three embodiments of the indoor navigation device with the improvements described herein. First, FIG. 7A is a front view of an improved mobile device 201 having a wrist mounting strap 211. FIG. 7B is a front view of device 202 with a chest mounting strap 212. Both the wrist mounted device 201 and the chest mounted device 202 are intended to be in contact with the skin of the wearer for the reasons to be explained herein. FIG. 7C is the front view of a device 203 for mounting on the helmet of a construction worker or other person. Device 203 can be mounted on a helmet in a number of widely known ways, for example by Velcro fasteners or by the use of adhesives. Each of the devices 201, 201, and 203 have four control buttons that have the functionality, starting at the bottom left corner of each device and proceeding clockwise, Start, Reset, Scan, and Configure (CNFG).

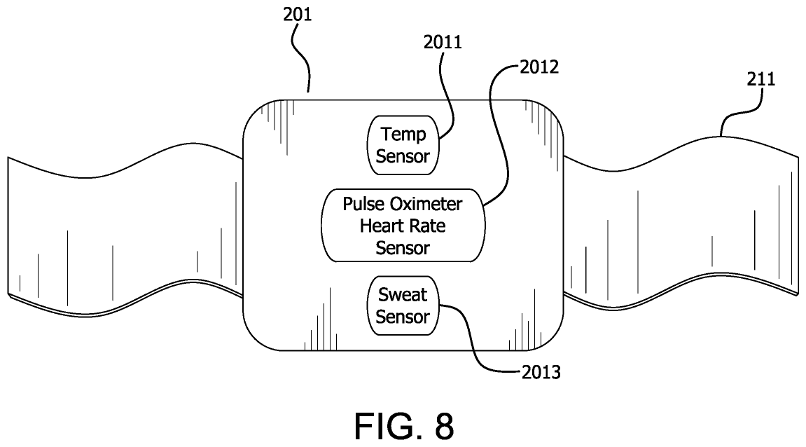

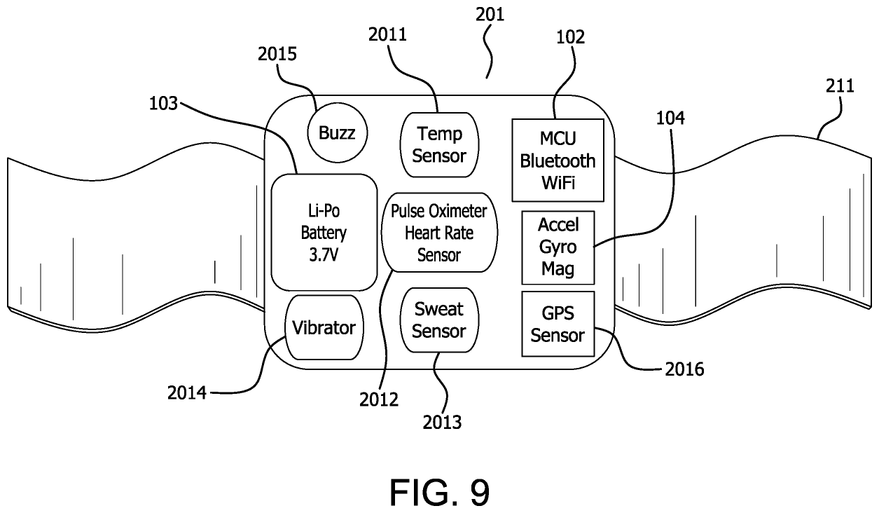

FIG. 8 is a block diagram showing certain components of the device 201 of FIG. 7A which are used to determine, record, and monitor vital signs of the wearer of the wrist mounted device 201. Each of the temperature sensor 2011, the pulse oximeter and heart rate sensor 2012, and the sweat sensor 2013 are positioned on a wearable device, such as wrist mounted device 201 or chest mounted device 202, to touch the skin of the person being monitored in order to provide an accurate and ongoing assessment of that person's body temperature, oxygen level, heart rate, and perspiration level. Changes in perspiration level can signal anxiety of a wearer who comes in close proximity to others or other issues with anxiety. As shown in FIG. 9, those vital sign components are used to provide additional functionality to the IND 201. The block diagram of the improved IND device 201 as shown in FIG. 9 includes certain IND components—MCU unit 102; 9-axis inertial measurement unit 104; and battery 104—along with the vital sign sensors 2011, 2012, and 2013 referenced above and additional components buzzer 2015 and vibrator 2014 that provide notification or alarms to the wearer of device 201 relating to the close proximity of others or of problematic deviations in vital signs, such as body temperature above a preset ceiling, such as 100° F. or oxygen saturation level below a preset floor, such as 90%. Additionally, the block diagram of FIG. 9 shows optional component 2016, a built in GPS sensor, that provides additional functionality to monitor and record vital signs out of doors, as well as inside a targeted real property.

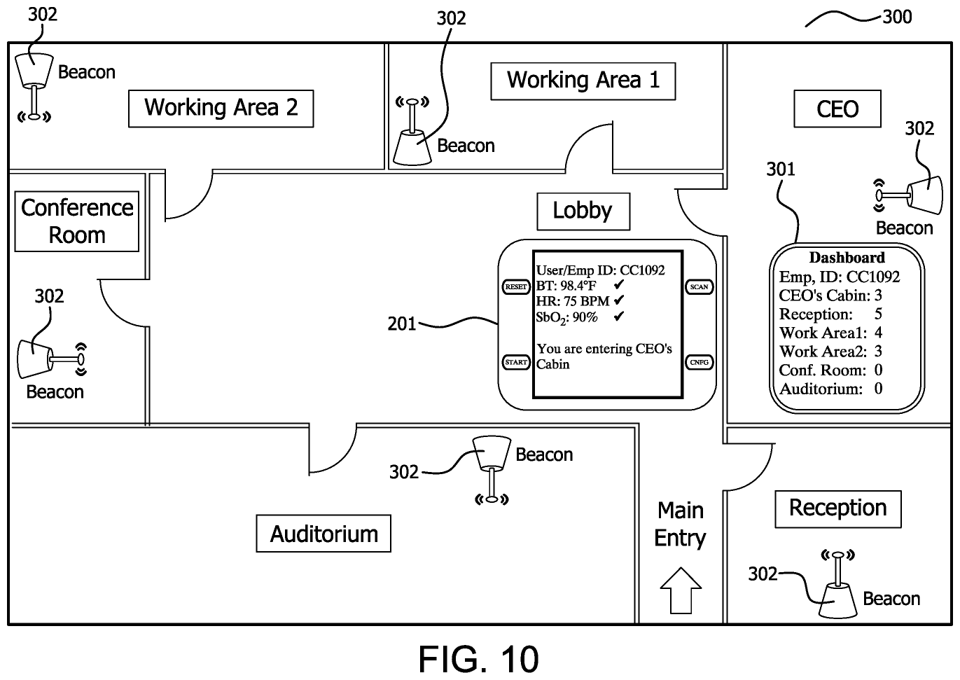

FIG. 10 shows a floor plan of a building fully outfitted for use with the preferred embodiment of the instant system. In one embodiment, the floor plan 300 shows beacons 302 located in various rooms or areas of the building being monitored, such as the reception area, the office of the Chief Executive Officer, or certain working areas. In such an embodiment, each of the beacons 302 transmit RF signals at determined intervals that identify location and, in one embodiment, can communicate with each device 201 that is then located in the subject property 300. In FIG. 10 an employee with an ID number CC 1092 wearing a skin touching, wrist mounted device 201 has entered the lobby of the building being monitored. The display of device 201 indicates the vital signs of that employee (within the acceptable ranges, that is, body temperature of 98.4° F., heart rate of 75 beats per minute, and oxygen level of 90%) and also indicates to the wearer that the employee is about to enter the office of the Chief Executive Officer in which is shown electronic dashboard 301 displayed on the CEO's laptop or smartphone that is used by management to monitor activity in the targeted building, as current locational and vital sign information collected by each of such devices 201 is transmitted wirelessly to the computing device, such as a server, that. The laptop, smartphone, or other computing device on which dashboard 301 is displayed and that is located in the CEO office is wirelessly connected to a computing device, such as a server, that monitors wirelessly the personal proximity device 201 worn by employee CC1092, as well as similar devices in the possession of other employees, or others, such as visitors to the property, as determined by management. Dashboard 301 indicates to the CEO that employee CC 1092 is about to enter the CEO's office another and also provides at a glance the number of persons then occupying each of the areas of the floor of the building, for example, the CEO can see that there are five persons in the reception area at the time in question.

As can be appreciated, disclosure of the system and method as set forth herein should not be viewed as limited to a preferred embodiment, not to any embodiments, uses, or types of properties as defined herein, but can be used in a variety of applications and uses. This disclosure is not limited to the specific embodiments as described herein.

What is claimed is:

1. A system for tracking and recording positional and health related information of persons at a targeted property comprising:
a plurality of portable electromechanical devices, each having wireless capability and each being in the possession of a person in or on said property;
at least one computing device having a screen display on which is displayed a plan of said property that is generated by and controlled by a software application running thereon, said computing device being in wireless communication with each of said electromechanical devices,
whereby the position of each of said persons having possession of said electromechanical device is represented by an electronically displayed marker on said plan displayed on said computing device, which marker automatically changes its position on said displayed plan matching any change in said position of said person; and
whereby the proximity of each of said persons to each of said other persons is tracked continuously and recorded.

2. The system of claim 1 in which said electronic computing device is selected from the group comprising: a laptop, a tablet computer, a mainframe computer, a handheld electronic device, and a smartphone.

3. The system of claim 1 in which each of said electromechanical devices comprises:
a circuit board, by which are connected the following electronic components:
a user switch;
a controller chip;
a self-contained power source;
two or more light signaling elements;
two or more crystal oscillators; and
an inertial measurement unit.

4. The system of claim 3 in which said self-contained power source is a coin cell battery.

5. The system of claim 3 in which said controller chip is a wireless microcontroller unit selected from the group of units in the model CC26XX family.

6. The system of claim 3 in which said light signaling elements are at least one red color light emitting diode and at least one green color light emitting diode.

7. The system of claim 3 in which said crystal oscillators are at least one 24 MHz crystal oscillator and at least one 32.768 kHz crystal oscillator.

8. The system of claim 3 in which said inertial measurement unit comprises:
a three axis accelerometer;
a three axis gyroscope; and
a three axis magnetometer.

9. The system of claim 8 in which said inertial measurement unit further comprises:
a digital motion processor.

10. The system of claim 9 in which said inertial measurement unit is an MPU9250 chip.

11. The system of claim 3 in which said electromechanical device further comprises at least one of the group of components comprising the following: a screen display; a body temperature sensor; a blood oxygen sensor; a heartbeat sensor; and a sweat sensor.

12. The system of claim 3 in which said electromechanical device further comprises a body temperature sensor, a blood oxygen sensor, a heartbeat sensor, and a sweat sensor, whereby the vital signs of each of said persons is tracked continuously and recorded.

13. A wearable electromechanical device for tracking locational and health information of the wearer comprising:
a circuit board, by which are connected the following electronic components:
a user switch;
a controller chip;
a self-contained power source;
two or more light signaling elements;
two or more crystal oscillators; and
an inertial measurement unit;
wireless circuitry; and
at least one of the group of component comprising the following: a body temperature sensor; a blood oxygen sensor; a heartbeat sensor; and a sweat sensor.

14. The wearable electromechanical device of claim 13 in which said self-contained power source is a coin cell battery.

15. The wearable electromechanical device of claim 13 which said controller chip is a wireless microcontroller unit selected from the group of units in the model CC26XX family.

16. The wearable electromechanical device of claim 13 in which said light signaling elements are at least one red color light emitting diode and at least one green color light emitting diode.

17. The wearable electromechanical device of claim 13 in which said crystal oscillators are at least one 24 MHz crystal oscillator and at least one 32.768 kHz crystal oscillator.

18. The wearable electromechanical device of claim 13 in which said inertial measurement unit comprises:
a three axis accelerometer;
a three axis gyroscope; and
a three axis magnetometer.

19. The wearable electromechanical device of claim 18 in which said inertial measurement unit further comprises:
a digital motion processor.

20. The wearable electromechanical device of claim 19 in which said inertial measurement unit is an MPU9250 chip.

21. A method for tracking and recording positional and health related information of persons located at targeted real property comprising the steps of:
scanning hardcopy plans for said property into uploadable electronic files;
labelling said electronic files;
uploading said electronic files into a computing device having a screen display and wireless capability, said computing device running under the control of an application program;
providing a plurality of wearable electromechanical devices into the possession of each of said persons;
having each of said persons wear each one of said electromechanical devices in a prescribed manner;
each of said electromechanical devices communicating wireless with said computing device;
said program calibrating said files with locations of said persons at said property as determined by the positioning of each of said electromechanical devices at said property;
displaying the position of and proximity to each of said persons on the screen display of said computing device on said plan of said property location;
tracking said positions and proximities; and
recording positional and health related information of each of said persons,
whereby the locations of, the proximities to, and the health related information of each of said persons is tracked and recorded.

22. The method of claim 21 in which said hardcopy plans are selected from the group comprising: surveys, floorplans, blueprints, and construction plans.

23. The method of claim 21 in which said electromechanical devices comprise:
a circuit board, by which are connected the following electronic components:
a user switch;
a controller chip;
a self-contained power source;
two or more light signaling elements;
two or more crystal oscillators; and
an inertial measurement unit;
wireless circuitry; and
at least one of the group of component comprising the following: a body temperature sensor; a blood oxygen sensor; a heartbeat sensor; and a sweat sensor.

24. The method of claim 23 in which said self-contained power source is a coin cell battery.

25. The method of claim 23 which said controller chip is a wireless microcontroller unit selected from the group of units in the model CC26XX family.

26. The method of claim 23 in which said light signaling elements are at least one red color light emitting diode and at least one green color light emitting diode.

27. The method of claim 23 in which said crystal oscillators are at least one 24 MHz crystal oscillator and at least one 32.768 kHz crystal oscillator.

28. The method of claim 23 in which said inertial measurement unit comprises:
a three axis accelerometer;
a three axis gyroscope; and
a three axis magnetometer.

29. The method of claim 28 in which said inertial measurement unit further comprises:
a digital motion processor.

30. The method of claim 29 in which said inertial measurement unit is an MPU9250 chip.
